(19) United States
(12) Patent Application Publication
(10) Pub. No.: US 2025/0304518 A1
(43) Pub. Date: Oct. 2, 2025

(54) PROCESS FOR PREPARATION OF ETHER, THIOETHER OR SECONDARY AMINE DERIVATIVES IN THE PRESENCE OF A HETEROGENEOUS ACIDIC CATALYST

(71) Applicant: Firmenich SA, Satigny (CH)

(72) Inventor: Pierre-Yves DAPSENS, Satigny (CH)

(21) Appl. No.: 18/873,464

(22) PCT Filed: Jun. 29, 2023
(86) PCT No.: PCT/EP2023/067862

§ 371 Date: Dec. 10, 2024

(30) Foreign Application Priority Data
Jun. 30, 2022 (EP) 22182094.7

(51) Int. Cl. C07C 41/03; C07C 67/16

(52) U.S. Cl. CPC C07C 41/03; C07C 67/16

(57) ABSTRACT

Disclosed herein is a process for the preparation of an ether, thioether or secondary amine of formula (I) including a reaction of an alcohol, thiol or amine of formula (II) with an epoxide of formula (III) performed in the presence of a heterogeneous acidic catalyst.

### TECHNICAL FIELD OF THE INVENTION

The present invention relates to the field of organic synthesis and, more specifically, it concerns a process for the preparation of an ether, thioether or secondary amine of formula (1) comprising the reaction of an alcohol, thiol or amine of formula (II) with an epoxide of formula (III) performed in the presence of a heterogeneous acidic catalyst.

### BACKGROUND OF THE INVENTION

The ether, thioether or secondary amine derivatives represent highly desirable skeletons which could be used as such or as key intermediates useful to prepare more complex compounds in different fields such as, among others, perfumery, cosmetic, pharmaceutic or agrochemistry. A relevant ether derivative is for example 2-((3,3-dimethylcyclohexyl)methoxy)-2-methylpropan-1-ol, which is used for the synthesis of Helvetolide® (trademark from Firmenich SA, Suisse), representing one of the most sought-after ingredients in the perfumery industry.

However, the possibilities of reaction pathways for the synthesis of Helvetolide® are limited and passes through 2-((3,3-dimethylcyclohexyl)methoxy)-2-methylpropan-1-ol as a key intermediate. Conventionally, α-3,3-Trimethylcyclohexanemethanol is reacted with 1,2-epoxy-2-methylpropan in the presence of a homogeneous acidic catalyst, e.g. a soluble Lewis acid. Unfortunately, the selectivity and consequently the conversion efficiency (yield) of this reaction is low. This lack of efficiency mainly originates from unwanted side reactions comprising ring opening reactions of α-3,3-Trimethylcyclohexanemethanol and formation of heavier products coming from the reaction of 1,2-epoxy-2-methylpropan with the 2-((3,3-dimethylcyclohexyl)methoxy)-2-methylpropan-1-ol in-situ generated. In addition, homogeneous catalysts suitable for this reaction can as well be corrosive for the equipment used for performing the reaction. Furthermore, the use of homogeneous catalysts that require a basic post-reaction washing step appeared particularly problematic owing to the nature of 2-((3,3-dimethylcyclohexyl)methoxy)-2-methylpropan-1-ol, which behaves as surfactant.

Consequently, there is a need to develop a reaction process with improved conversion efficiency and selectivity and therefore an improved yield and with a simplified workup procedure.

The present invention allows solving the above problem by using a heterogenous acidic catalyst in order to prepare an ether, thioether or secondary amine of formula (1). To the best of our knowledge, the invention's conditions have never been reported in the prior art.

### DETAILED DESCRIPTION OF THE INVENTION

Surprisingly, it has now been discovered that the preparation of a compound of formula (I) by reaction of a compound of formula (II) with an epoxide of formula (III) in presence of a heterogeneous acidic catalyst allows preparing a compound of formula (I) with high yield and high selectivity.

Therefore, a first object of the present invention is a process for the preparation of a compound of formula (I)

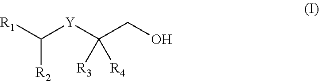

in the form of any of its stereoisomers or a mixture thereof; wherein
Y is a sulfur atom, an oxygen atom or a NH group, and
R₁ represents a C₁₋₁₂ alkyl, C₂₋₁₂ alkenyl, a C₅₋₁₂ cycloalkyl or C₅₋₁₂ cycloalkenyl group, each optionally substituted by 1 to 3 C₁₋₄ alkyl groups; and
each R₂, R₃, R₄ represent, when taken separately, independently from each other, a hydrogen atom or a C₁₋₄ alkyl group;
comprising the reaction of a compound of formula (II)

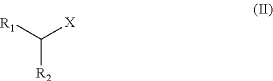

wherein X is a thiol group, alcohol group or primary amine group, and
wherein R₁ and R₂ have the same meaning as defined above;
with an epoxide of formula (III)

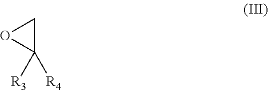

wherein R₃ and R₄ have the same meaning as defined above;
in the presence of a heterogeneous acidic catalyst.

For the sake of clarity, by the expression “any one of its stereoisomers or a mixture thereof”, or the similar, it is meant the normal meaning understood by a person skilled in the art, i.e. that the compounds cited in the invention can be a pure enantiomer or a mixture of enantiomers. In other words, the compounds cited in the invention may possess at least one stereocenter which can have two different stereochemistries (e.g. R or S), e.g. the R¹ group may comprise at least one stereocenter. Said compounds may even be in the form of a pure enantiomer or in the form of a mixture of enantiomers. The compounds cited in the invention may even be in the form of a pure diastereoisomer or in the form of a mixture of diastereoisomers when said compounds possess more than one stereocenter. Said compounds can be in a racemic form or scalemic form. Therefore, said compounds can be one stereoisomer or in the form of a composition of matter comprising, or consisting of, various stereoisomers.

It is understood that if Y is a sulfur atom, the compound comprises a thiolether group.

It is understood that if Y is an oxygen atom, the compound comprises an ether group.

It is understood that if Y is a NH group, the compound comprises a secondary amine group.

The term “optionally” is understood that a group can or cannot comprise a certain functional group or substituent.

The term “alkyl group” is understood as comprising linear or branched alkyl groups.

The term “alkenyl group” is understood as comprising linear or branched alkenyl groups.

The expression “C₁₋₁₂ alkyl, C₂₋₁₂ alkenyl, a C₅₋₁₂ cycloalkyl or C₅₋₁₂ cycloalkenyl group, each optionally substituted by 1 to 3 C₁₋₄ alkyl groups” is understood as the cycloalkyl or the cycloakenyl being optionally substituted by 1 to 3 C₁₋₄ alkyl groups.

According to a particular embodiment, X is an alcohol group and Y is an oxygen atom.

According to a particular embodiment, R₁ is a C₁₋₁₀ alkyl, C₂₋₁₀ alkenyl, a C₅₋₁₀ cycloalkyl or C₅₋₁₀ cycloalkenyl group, each optionally substituted by 1 to 3 C₁₋₄ alkyl groups. Particularly, R₁ is a C₁₋₃ alkyl, C₂₋₈ alkenyl, a C₅₋₁₀ cycloalkyl or C₅₋₁₀ cycloalkenyl group, each optionally substituted by 1 to 3 C₁₋₄ alkyl groups. Particularly, R₁ is a C₁₋₆ alkyl, C₂₋₆ alkenyl, a C₅₋₁₀ cycloalkyl or C₅₋₁₀ cycloalkenyl group, each optionally substituted by 1 to 3 C₁₋₄ alkyl groups. Particularly, R₁ is a C₅₋₁₀ cycloalkyl or C₅₋₁₀ cycloalkenyl group, preferably a C₅₋₈ cycloalkyl or C₅₋₈ cycloalkenyl group, preferably a C₅₋₇ cycloalkyl or C₅₋₇ cycloalkenyl group, preferably a C₅₋₆ cycloalkyl or C₅₋₆ cycloalkenyl group, more preferably a C₆ cycloalkyl group, each optionally substituted by 1 to 3 C₁₋₄ alkyl groups, preferably by 1 to 2 C₁₋₃ alkyl groups, even more preferably by 1 to 2 C₁₋₂ alkyl groups. Even more particularly, R₁ is a 3,3-dimetly-1-cyclohexyl group.

According to a particular embodiment, R² represents a hydrogen atom or a C₁₋₃ alkyl group. Particularly, R² represents a methyl or ethyl group, more preferably a methyl group.

According to a particular embodiment, R³ represents a hydrogen atom or a C₁₋₃ alkyl group. Particularly, R³ represents a methyl or ethyl group, more preferably a methyl group.

According to a particular embodiment, R⁴ represents a hydrogen atom or a C₁₋₃ alkyl group. Particularly, R⁴ represents a methyl or ethyl group, more preferably a methyl group.

According to a more particular embodiment, each R³ and R⁴ represent a methyl group.

According to a particular embodiment, the present invention is directed to a process, wherein the compound of formula (II) is of formula (IIa)

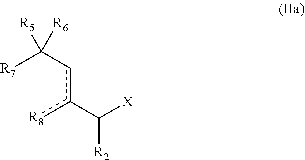

in the form of any of its stereoisomers or a mixture thereof,
wherein
one dotted line is a carbon-carbon single or double bond and the other is a carbon-carbon single bond; and
X is a thiol group, alcohol group or primary amine group, and
each R₂, R₅, R₆, R₇ and R₈ represent, when taken separately, independently from each other, a hydrogen atom or a C₁₋₄ alkyl group; or
R₇ and R₈ are linked to each other and form a C₅₋₇ cycloalkyl or cycloalkenyl group; and the compound of formula (I) is of formula (Ia)

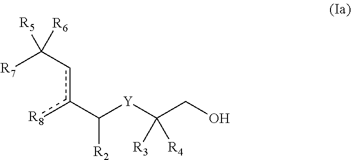

in the form of any of its stereoisomers or a mixture thereof,
wherein
Y is a sulfur atom, an oxygen atom or a NH group, and
the dotted line, R₂, R₅, R₆, R₇ and R₈ have the same meaning as defined above; and
each R₃ and R₄ represent, when taken separately, independently from each other, a hydrogen atom or a C₁₋₄ alkyl group.

For the sake of clarity, by the expression “one dotted line is a carbon-carbon single or double bond and the other is a carbon-carbon single bond” it is meant the normal meaning understood by a person skilled in the art, i.e. that the whole bonding (solid and dotted line) between the carbon atoms connected by said dotted line is a carbon-carbon single or double bond.

It is understood by a person skilled in the art that in case R₈ represents a hydrogen atom, the dotted line between R₈ and the neighboring carbon atom represents a hydrogen-carbon single bond.

For the sake of clarity, by the expression “in case R₇ and R₈ are linked to each other, they form a C₅₋₇ cycloalkyl or cycloalkenyl group” it is meant that R₇ and R₈ can be chemically connected via a carbon-carbon single or double bond forming a C₅₋₇ cycloalkyl or cycloalkenyl group comprising the other carbon atoms of the structure.

According to particular embodiments, R² to R³ have the same meanings as defined herein above.

According to a particular embodiment, the compound of formula (IIa) is of formula (IIa,i)

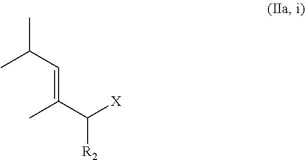

in the form of any of its stereoisomers or a mixture thereof,
wherein
X is a thiol group, alcohol group or primary amine group, and
R² a hydrogen atom or a C₁₋₄ alkyl group; and
the compound of formula (Ia) is of formula (Ia,i)

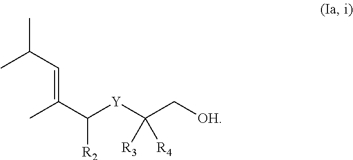

in the form of any of its stereoisomers or a mixture thereof,
wherein
Y is a sulfur atom, an oxygen atom or a NH group, and
each R², R³, R⁴ represent, when taken separately, independently from each other, a hydrogen atom or a C₁₋₄ alkyl group.

According to a particular embodiment, the present invention is directed to a process, wherein the compound of formula (II) is of formula (IIb)

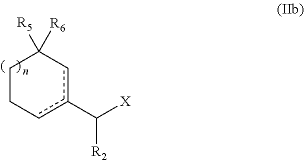

in the form of any of its stereoisomers or a mixture thereof,
wherein
n is 0 or 1;
X is a thiol group, alcohol group or primary amine group, and
one dotted line is a carbon-carbon single or double bond and the other is a carbon-carbon single bond; and
each R₂, R₅ and R₆ represent, when taken separately, independently from each other, a hydrogen atom or a C₁₋₄ alkyl group; and
the compound of formula (I) is of formula (Ib)

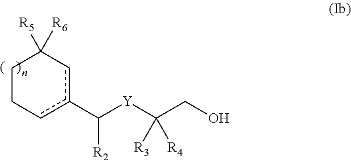

in the form of any of its stereoisomers or a mixture thereof,
wherein
Y is a sulfur atom, an oxygen atom or a NH group, and
the dotted line, n, R₂, R₅ and R₆ have the same meaning as defined above; and
each R₃ and R₄ represent, when taken separately, independently from each other, a hydrogen atom or a C₁₋₄ alkyl group.

According to particular embodiments, n is 1.

According to particular embodiments, R² to R⁶ have the same meanings as defined herein above.

According to a particular embodiment, the present invention is directed to a process, wherein the compound of formula (II) is of formula (IIc)

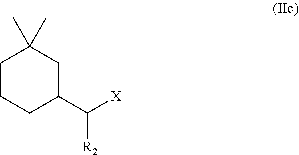

in the form of any of its stereoisomers or a mixture thereof,
wherein
X is a thiol group, alcohol group or primary amine group, and
R² a hydrogen atom or a C₁₋₄ alkyl group; and
the compound of formula (I) is of formula (Ic)

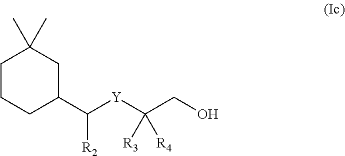

in the form of any of its stereoisomers or a mixture thereof,
wherein
Y is a sulfur atom, an oxygen atom or a NH group, and
each R², R³, R⁴ represent, when taken separately, independently from each other, a hydrogen atom or a C₁₋₄ alkyl group.

According to particular embodiments, R² to R⁴ have the same meanings as defined herein above.

According to a more particular embodiment, the compound of formula (I) is of formula (Id)

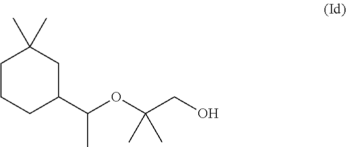 [SMILES: CC(OC(C)(C)CO)C1CCCC(C)(C)C1]

in the form of any of its stereoisomers or a mixture thereof; and the compound of formula (II) is of formula (IId)

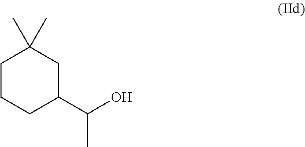

in the form of any of its stereoisomers or a mixture thereof, and the epoxide of formula (III) is of formula (IIId)

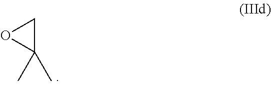

### Heterogeneous Acidic Catalyst

According to any embodiment of the present invention, the heterogeneous acidic catalyst may be amorphous or crystalline, particularly crystalline.

According to any embodiment of the present invention, the heterogeneous acidic catalyst may comprise at least one metal selected from the group consisting of silicon, tin, zirconium, hafnium or titanium or a mixture thereof and optionally at least one metal selected from the group consisting of aluminum, boron, iron or a mixture thereof. Preferably, the heterogeneous acidic catalyst comprises at least one metal selected from the group consisting of silicon or tin or a mixture thereof and optionally at least one metal selected from the group consisting of aluminum, boron, iron or a mixture thereof.

According to any embodiment of the present invention, the heterogeneous acidic catalyst may be a Lewis acid supported on a solid support.

A person skilled in the art is able to select suitable Lewis acids. Suitable Lewis acids may be HClO₄, BF₃, AlCl₃, FeCl₃, CuCl₂, ZnCl₂, ZnBr₂, ZrCl₄, TiCl₄ or TiCl₄₋ₓ(OR)ₓ, wherein R represents a C₁₋₁₂ alkyl or alkenyl group. Other suitable Lewis acids may be metal trifluoromethanesulfonates, wherein the metal may be a metal selected from the series of lanthanides, preferably lanthanum. Other suitable Lewis acids may be heteropolyacids (also called heteropolymetalates). A person skilled in the art is able to select suitable heteropolyacids based on his general knowledge in the field. Preferred heteropolyacids are silicotungstic acid (H₄SiW₁₂O₄₀·nH₂O), phosphomolybdic acid (H₃Mo₁₂PO₄₀·nH₂O) or phosphotungstic acid (H₃W₁₂PO₄₀·nH₂O).

According to an embodiment, the supported Lewis acid can be a mixture of two or more Lewis acids.

The solid support is understood to remain solid under the chosen reaction conditions. According to a particular embodiment, the solid support may be an organic or inorganic polymer. According to a particular embodiment, the solid support may be an inorganic support material. According to a particular embodiment, the inorganic support material may be a metal oxide. According to a particular embodiment, the inorganic support material may comprise a metal selected from the group consisting of silicon, tin, aluminum, zirconium, titanium, iron, boron or a mixture thereof, preferably silicon. According to a particular embodiment, the inorganic support material may be silicon dioxide. According to a particular embodiment, the supported Lewis acid is BF₃/SiO₂ or HClO₄/SiO₂.

According to any embodiment of the present invention, the heterogeneous acidic catalyst is an aluminosilicate catalyst.

According to any embodiment of the present invention, the heterogeneous acidic catalyst is a zeolite or a clay.

According to any embodiment of the present invention, the heterogeneous acidic catalyst is a clay.

The clay may be a commercially available clay. According to any embodiment of the present invention, the commercially available clay may contain water. Said water may be removed partly or totally before use. The person skilled in the art is well-aware of methods to remove water such as, for example, azeotropic distillation, vacuum striping or heating under nitrogen flow.

According to any embodiment of the present invention, the clay may be naturally occurring. According to any embodiment of the present invention, the heterogeneous acidic catalyst is an acid treated clay. An acidic treated clay is herein understood to be a clay that is subjected to an acidic treatment prior to its use as heterogeneous acidic catalyst according to the methods described in the U.S. Pat. Nos. 2,470,872A, 2,671,058A, 2,981,697A or 1,926,148A. A person skilled is able to select the most appropriate conditions for the acid treatment of the clay based on his general knowledge in the field and on the teaching of these patent applications. Non-limiting examples of suitable clays may include type K clays such as K-5, K10-S300, K-20, K-30, K-41 or K-306 (presently sold by Clariant), former Filtrol type clays such as F-20X, F20-XLM, F-21X, F-24X, F-25X, F-31X, F-54X, F-22, or F-118FF (presently sold by EP Minerals), Fulcat-22F, Fulcat-22B or Fulcat 435 (presently sold by Byk), EXBC 0001 (presently sold by Clariant). In an embodiment, the clay is K10-S300.

According to a preferred embodiment of the present invention, the heterogeneous acidic catalyst is a zeolite.

According to any embodiment of the present invention, the zeolite is used in its protonic form. The latter can be directly provided by the manufacturer and used as such or obtained by thermal decomposition of the ammonium-exchange form. In a particular embodiment, the zeolite is a preactivated zeolite. The preactivation may be carry out by removing trapped water as described earlier in the case of clays or by heating the zeolite at a temperature comprised between 300° C. and 600° C. for at least 1 hour under air or an inert gas.

The heterogeneous acidic catalyst is commercially available compound or can be prepared by several methods, such as the one reported in US20040141911, U.S. Pat. Nos. 6,054,113, 4,840,930, 2,470,872 and EP0398636.

Non-limiting examples of suitable zeolites may include CBV780, CBV901 presently sold by Zeolyst or HSZ-385HUA or HSZ-390HUA presently sold by Tosoh.

According to any embodiment of the present invention, the heterogeneous acidic catalyst is a large pore zeolite.

By “the term large pore zeolite” it is meant the normal meaning in the art; i.e. a 12 membered ring zeolite having a pore size comprised in the range between 6.0 Angstrom and 7.5 Angstrom. Non-limiting examples of suitable large pore zeolite may include FAU, BEA, MOR.

According to any embodiment of the present invention, the heterogeneous acidic catalyst is a zeolite having a FAU topology.

The term FAU topology is understood to have the meaning conventionally used in the field of zeolites and are well-known to a person skilled in the art. The framework topology is usually defined by a three letters code following the rules set up by an IUPAC Commission on Zeolite Nomenclature in 1978 (R. M. Barrer, Pure Appl. Chem. 51, 1091 (1979)).

A zeolite having a FAU topology is understood as zeolite with a faujasite crystal structure. A person skilled in the art is aware of the definition of the faujasite crystal structure, which is e.g. described in Römpp Chemie Lexikon, Georg Thieme Verlag, 9. Edition, 1990, page 1311

According to any embodiment of the present invention, the heterogeneous acidic catalyst is a Y-type zeolite.

The term Y-type zeolite is understood to have the meaning conventionally used in the field of zeolites and is well-known to a person skilled in the art. A Y-type zeolite is understood to be a zeolite having a FAU topology. A Y-type zeolite is typically characterized by spherical, internal cavities (so called “supercages”) linked tetrahedrally through pore openings of about 8 Angstrom and comprising rings of twelve oxygen atoms.

According to any embodiment of the present invention, the heterogeneous acidic catalyst is a dealuminated zeolite.

Dealumination is conventionally understood as removal of aluminum atoms from a zeolite structure. Dealumination leads to an increase of the Silicon:Aluminum ratio of a zeolite. Non-limiting examples for suitable methods for dealumination known in the art are hydrothermal treatment, acidic treatment, treatment with gaseous halides or halogens or complexation with chelating agents. A person skilled in the art is aware of these methods and of ways to perform them.

According to any embodiment of the present invention, the heterogeneous acidic catalyst is a dealuminated ultrastable Y-type (USY) zeolite.

USY zeolites are typically prepared from Y-type zeolites to increase their stability and improve their catalytic activity by removing intra-framework aluminum with a combination of treatments comprising ion-exchange, steaming, acid leaching and calcination. Such treatments are for example described in the U.S. Pat. Nos. 5,601,798A and 4,477,336A and are well-known to a person skilled in the art.

According to any embodiment of the present invention, the heterogeneous acidic catalyst is a hydrophobic zeolite.

According to any embodiment of the present invention, the heterogeneous acidic catalyst is a hydrophobic dealuminated USY zeolite.

Hydrophobicity of zeolites is conventionally defined by the hydrophobicity index (HI). The hydrophobicity index is defined as the amount (usually in grams) of cyclohexane adsorbed per weight (usually in grams) of water adsorbed on a unit amount of zeolite at vapor pressures of cyclohexane and water of 933 Pa (7 Torr) and 666 Pa (5 Torr), respectively, according to I. Halasz et al., Molecular Physics, 2002, 100, 3232-3232.

According to any embodiment of the present invention, the hydrophobicity index is between 5-100, preferably between 10 and 75 and more preferably between 20 and 50.

According to any embodiment of the present invention, the Silicon:Aluminum ratio is in the range between 5:1 and 350:1.

According to a preferred embodiment, the Silicon:Aluminum ratio is in the range between 10:1 and 325:1.

According to a preferred embodiment, the Silicon:Aluminum ratio is in the range between 15:1 and 300:1.

According to a preferred embodiment, the Silicon:Aluminum ratio is in the range between 22:1 and 275:1.

According to a preferred embodiment, the Silicon:Aluminum ratio is in the range between 30:1 and 250:1.

According to a preferred embodiment, the Silicon:Aluminum ratio is in the range between 40:1 and 250:1.

According to a preferred embodiment, the Silicon:Aluminum ratio is in the range between 50:1 and 250:1.

In a particular embodiment, the heterogeneous acidic catalyst is selected from the group consisting of clay, zeolite, and supported Lewis acid. The clay, zeolite, and supported Lewis acid, respectively, can be as defined above.

The heterogeneous acidic catalyst can be added into the reaction medium of the invention's process to form the compound of formula (I) in a large range of concentrations.

According to any embodiment of the present invention, the heterogeneous acidic catalyst is used in an amount of 2 to 50 wt. %, preferably 3 to 30 wt. %, more preferably 4 to 15 wt. %, even more preferably 5 to 10 wt. %, relative to the amount of epoxide of formula (III).

According to any embodiment of the present invention, the compound of formula (II) or (IIa) is used in an amount of 1.0 to 10 equivalents, preferably in an amount of 1.4 to 7 equivalents, more preferably in an amount of 1.8 to 4.2 equivalents, relative to the amount of epoxide of formula (III).

According to any embodiment of the present invention, the process temperature is kept in a range between 0 to 50° C., preferably between 25 and 48° C., more preferably between 43 and 47° C. A person skilled in the art is able to select the preferred temperature as a function of the melting and boiling point of the starting and final products as well as the desired time of reaction, conversion or selectivity.

The invention's process for the preparation of a compound of formula (I) can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene pseudocumene, anisole or chlorobenzene or mixtures thereof, hydrocarbon solvents such as cyclohexane, heptane or mixtures thereof, nitrile solvent such as acetonitrile, or ethereal solvents such as tetrahydrofuran, diethyether, methyl tetrahydrofuran or mixtures thereof. The choice of the solvent is function of the nature of the substrate and/or catalyst and the person skilled in the art is well able to select the solvent most suitable in each case to optimize the reaction.

The invention's process for the preparation of a compound of formula (I) may be carried out under batch and/or continuous conditions.

A process according to any embodiment of the present invention can be used for the preparation of a compound of formula (IV)

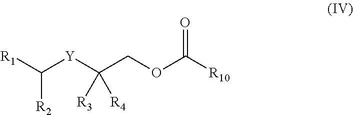

in the form of any of its stereoisomers or a mixture thereof; wherein
Y is a sulfur atom, an oxygen atom or a NH group, and
R₁ represents a C₁₋₁₂ alkyl, C₂₋₁₂ alkenyl, a C₅₋₁₂ cycloalkyl or C₅₋₁₂ cycloalkenyl group, each optionally substituted by 1 to 3 C₁₋₄ alkyl groups; and
each R₂, R₃, R₄ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; preferably a C₁₋₄ alkyl group, a C₂₋₄ alkenyl group, a C₃₋₆ cycloalkyl group; preferably a C₁₋₃ alkyl group, a C₂₋₃ alkenyl group, a C₃₋₅ cycloalkyl group; preferably an ethyl group, a vinyl group, a cyclopropyl or a cyclopentyl group, more preferably an ethyl group;
by reacting the compound of formula (I) with a compound of formula Z—C(O)—R₁₀, wherein Z is a R₁₀—C(O)—O group, thiol group, chlorine atom, alcohol group or amine group.

According to any embodiment of the present invention, the compound of formula (IV) is of formula (IVa)

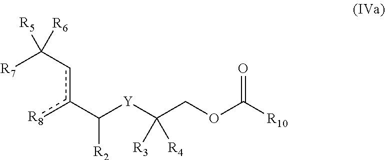

in the form of any of its stereoisomers or a mixture thereof; wherein
one dotted line is a carbon-carbon single or double bond and the other is a carbon-carbon single bond; and
Y is a sulfur atom, an oxygen atom or a NH group, and
each R₂, R₃, R₄, R₅, R₆ and R₇ represent, when taken separately, independently from each other, a hydrogen atom or a C₁₋₄ alkyl group; and
   R₈ represents a C₁₋₄ alkyl group; or
R₇ and R₈ are linked to each other and form a C₅₋₇ cycloalkyl or cycloalkenyl group; and
R₁₀ represents a C₁₋₆ alkyl group, a C₂₋₆ alkenyl group, a C₃₋₆ cycloalkyl group; preferably a C₁₋₄ alkyl group, a C₂₋₄ alkenyl group, a C₃₋₆ cycloalkyl group; preferably a C₁₋₃ alkyl group, a C₂₋₃ alkenyl group, a C₃₋₅ cycloalkyl group; preferably an ethyl group, a vinyl group, a cyclopropyl or a cyclopentyl group, more preferably an ethyl group; and
the compound of formula (IVa) is formed by reacting the compound of formula (Ia) with a compound of formula Z—C(O)—R₁₀, wherein Z is a R₁₀—C(O)—O group, thiol group, chlorine atom, alcohol group or amine group.

According to any embodiment of the present invention, the compound of formula (IV) is of formula (IVa,i)

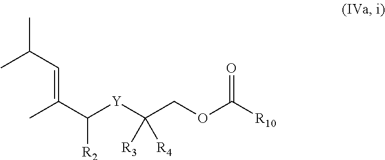

in the form of any of its stereoisomers or a mixture thereof; wherein
one dotted line is a carbon-carbon single or double bond and the other is a carbon-carbon single bond; and
Y is a sulfur atom, an oxygen atom or a NH group, and
each R₂, R₃ and R₄ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; preferably a C₁₋₄ alkyl group, a C₂₋₄ alkenyl group, a C₃₋₆ cycloalkyl group; preferably a C₁₋₃ alkyl group, a C₂₋₃ alkenyl group, a C₃₋₅ cycloalkyl group; preferably an ethyl group, a vinyl group, a cyclopropyl or a cyclopentyl group, more preferably an ethyl group; and
the compound of formula (IVa) is formed by reacting the compound of formula (Ia) with a compound of formula Z—C(O)—R₁₀, wherein Z is a R₁₀—C(O)—O group, thiol group, chlorine atom, alcohol group or amine group.

According to any embodiment of the present invention, the compound of formula (IV) is of formula (IVb)

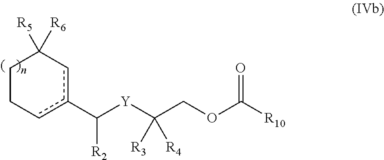

in the form of any of its stereoisomers or a mixture thereof; wherein
n is 0 or 1; and
one dotted line is a carbon-carbon single or double bond and the other is a carbon-carbon single bond; and
Y is a sulfur atom, an oxygen atom or a NH group, and
each R₂, R₃, R₄, R₅ and R₆ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; preferably a C₁₋₄ alkyl group, a C₂₋₄ alkenyl group, a C₃₋₆ cycloalkyl group; preferably a C₁₋₃ alkyl group, a C₂₋₃ alkenyl group, a C₃₋₅ cycloalkyl group; preferably an ethyl group, a vinyl group, a cyclopropyl or a cyclopentyl group, more preferably an ethyl group; and
the compound of formula (IVb) is formed by reacting the compound of formula (Ib) with a compound of formula Z—C(O)—R₁₀, wherein Z is a R₁₀—C(O)—O group, thiol group, chlorine atom, alcohol group or amine group.

According to any embodiment of the present invention, the compound of formula (IV) is of formula (IVc)

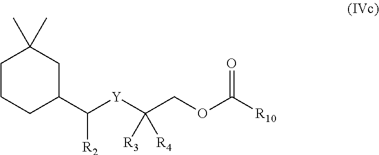

in the form of any of its stereoisomers or a mixture thereof; wherein
Y is a sulfur atom, an oxygen atom or a NH group, and
each R₂, R₃ and R₄ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; preferably an ethyl group, an ethylene group, a cyclopropane or a cyclopentane group, more preferably an ethyl group; and
the compound of formula (IVc) is formed by reacting the compound of formula (Ic) with a compound of formula Z—C(O)—R₁₀, wherein Z is a R₁₀—C(O)—O group, thiol group, chlorine atom, alcohol group or amine group.

According to a particular embodiment, R¹ represents a C₅₋₈ cycloalkyl optionally substituted by 1 to 2 C₁₋₃ alkyl groups, even more 1 to 2 C₁₋₂ alkyl groups, more preferably a C₆ cycloalkyl substituted by 2 methyl groups.

According to a particular embodiment, R² represents a methyl or ethyl group, more preferably a methyl group.

According to a particular embodiment, R³ represents a methyl or ethyl group, more preferably a methyl group.

According to a particular embodiment, R⁴ represents a methyl or ethyl group, more preferably a methyl group.

According to a more particular embodiment, each R³ and R⁴ represent a methyl group.

According to a particular embodiment, R⁵ represents a methyl or ethyl group, more preferably a methyl group.

According to a particular embodiment, R⁶ represents a methyl or ethyl group, more preferably a methyl group.

According to a more particular embodiment, each R⁵ and R⁶ represent a methyl group.

According to a particular embodiment, R⁷ represents a hydrogen atom.

According to a particular embodiment, R⁸ represents a methyl or ethyl group, more preferably a methyl group.

According to any embodiment of the present invention, the compound of formula (IV) is of formula (IVd)

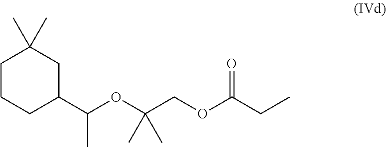 [SMILES: CCC(=O)OCC(C)(C)OC(C)C1CCCC(C)(C)C1]

in the form of any of its stereoisomers or a mixture thereof.

Typical manners to execute the invention's process are reported herein below in the examples, which should not be considered as limiting the invention. In the examples, unless otherwise specified, the abbreviations have the usual meaning in the art, the temperatures are indicated in degrees centigrade (° C.) and percentages are indicated in weight percent (wt. %).

### EXAMPLES

### Example 1: General Catalyst Screening Procedure

In a 0.5 L double jacketed glass reactor equipped with a mechanical stirrer, 130 g (0.832 mol) of (R)-1-((S)-3,3-dimethylcyclohexyl)ethan-1-ol (herein called Cyclademol™, origin DRT) were vigorously stirred at 45° C. in the presence of 2.08 g of catalyst (10 wt. % vs isobutylene oxide (IBO)). To this solution, 20.8 g (0.288 mol) of isobutylene oxide (IBO, origin BASF) were slowly added in two hours and the reaction left further under stirring for another two hours. After filtration of the catalyst, the yield of (S)-2-((3,3-dimethylcyclohexyl)methoxy)-2-methylpropan-1-ol vs IBO in the crude sample was evaluated by gas chromatography using n-decane as internal standard.

### Example 2: Zeolite Catalysts

Several zeolites as listed in table 1 were used as catalyst in the procedure of Example 1. The results of this screening are shown in table 1. Each zeolite in Table 1 was calcined prior to use.

TABLE 1
Freshly calcined zeolites

| | Framework | Si/Al | Yield vs. IBO |
|---|---|---|---|
| Catalyst | topology | ratio | (mol. %) |
| CBV901 calc | FAU | 40 | 37.56 |
| HSZ-385HUA calc | | 57 | 37.82 |
| HSZ-390HUA calc | | 250 | 36.58 |

### Example 3: As-Received USY Zeolites with Lower Catalyst Loading

In a 1 L double jacketed glass reactor equipped with a mechanical stirrer, 400 g (2.56 mol) of (R)-1-((S)-3,3-dimethylcyclohexyl)ethan-1-ol (herein called Cyclademol™, origin DRT) were vigorously stirred at 45° C. in the presence of 3.2 g of as-received catalyst (5 wt. % vs isobutylene oxide (IBO)). To this solution, 64 g (0.887 mol) of isobutylene oxide (IBO, origin BASF) were slowly added in two hours and the reaction left further under stirring for another two hours. After filtration of the catalyst, the yield of (S)-2-((3,3-dimethylcyclohexyl)methoxy)-2-methylpropan-1-ol vs. IBO in the crude sample was evaluated by gas chromatography using n-decane as internal standard.

TABLE 2
As-received USY zeolites

| | Framework | Si/Al | Yield vs. IBO |
|---|---|---|---|
| Catalyst | topology | ratio | (mol. %) |
| CBV901 | FAU | 40 | 31.4 |
| HSZ-385HUA | | 57 | 35.65 |
| HSZ-390HUA | | 250 | 37.91 |

### Example 4: Comparative Homogeneous Catalyst

In a 0.5 L double jacketed glass reactor equipped with a mechanical stirrer, 130 g (0.832 mol) of (R)-1-((S)-3,3-dimethylcyclohexyl)ethan-1-ol (herein called Cyclademol™, origin DRT) were vigorously stirred at 30° C. in the presence of 16.8 mmol of a homogenous catalyst as defined in Table 3 below. To this solution, 20.8 g (0.288 mol) of isobutylene oxide (IBO, origin BASF) were slowly added in two hours and the reaction left further under stirring for another hour. After quenching of the solution with aqueous sodium citrate, the yield of (S)-2-((3,3-dimethylcyclohexyl)methoxy)-2-methylpropan-1-ol vs. IBO in the crude sample was evaluated by gas chromatography using n-decane as internal standard.

TABLE 3
Homogenous catalysts

| | Mass of Catalyst | Yield vs. IBO |
|---|---|---|
| Catalyst | (g) | (mol. %) |
| BF₃OEt₂ | 2.39 | 26.8 |
| BF₃OAc₂ | 3.15 | 20.7 |
| FeCl₃ | 2.72 | 6.9 |
| AlCl₃ | 2.24 | 5.1 |
| La(OTf)₃ | 9.84 | 19.7 |

### Example 5: Preparation of 2-((3,5-dimethylhex-3-en-2-yl)oxy)-2-methylpropan-1-ol

14 g (0.109 mol) of 3,5-dimethylhex-3-en-2-ol (prepared according to the procedure described in WO2004/050595 A1— example 1) were vigorously stirred at 45° C. in the presence of 0.274 g of HSZ-390 HUA (Tosoh). To this solution, 2.74 g (0.038 mol) of isobutylene oxide (IBO, origin BASF) were slowly added in two hours and the reaction left further under stirring for another hour. After filtration of the solid catalyst, the yield of 2-((3,5-dimethylhex-3-en-2-yl)oxy)-2-methylpropan-1-ol vs. IBO in the crude sample was evaluated by gas chromatography using n-decane as internal standard and reached 42.3 mol. %.

1. A process for the preparation of a compound of formula (I) 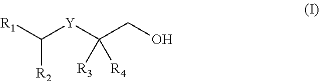
in the form of any of its stereoisomers or a mixture thereof, wherein
Y is a sulfur atom, an oxygen atom or a NH group, and
R₁ represents a C₁₋₁₂ alkyl, C₂₋₁₂ alkenyl, a C₅₋₁₂ cycloalkyl or C₅₋₁₂ cycloalkenyl group, each optionally substituted by 1 to 3 C₁₋₄ alkyl groups; and
each R₂, R₃, R₄ represent, when taken separately, independently from each other, a hydrogen atom or a C₁₋₄ alkyl group;
comprising a reaction of a compound of formula (II) 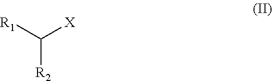
wherein X is a thiol group, alcohol group or primary amine group, and
wherein R₁ and R₂ have the same meaning as defined above;
with an epoxide of formula (III) 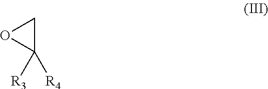
wherein R₃ and R₄ have the same meaning as defined above;
in the presence of a heterogeneous acidic catalyst.

2. The process according to claim 1, wherein the compound of formula (II) is of formula (IIa) 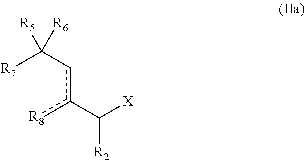
in the form of any of its stereoisomers or a mixture thereof,
wherein
one dotted line is a carbon-carbon single or double bond and the other is a carbon-carbon single bond; and
X is a thiol group, alcohol group or primary amine group, and
each R₂, R₅, R₆, R₇ and R₈ represent, when taken separately, independently from each other, a hydrogen atom or a C₁₋₄ alkyl group; or
R₇ and R₈ are linked to each other and form a C₅₋₇ cycloalkyl or cycloalkenyl group;
and
the compound of formula (I) is of formula (Ia) 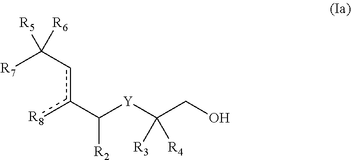
in the form of any of its stereoisomers or a mixture thereof,
wherein
Y is a sulfur atom, an oxygen atom or a NH group, and
the dotted line, R₂, R₅, R₆, R₇ and R₈ have the same meaning as defined above; and
each R₃ and R₄ represent, when taken separately, independently from each other, a hydrogen atom or a C₁₋₄ alkyl group.

3. The process according to claim 1, wherein the compound of formula (II) is of formula (IIc) 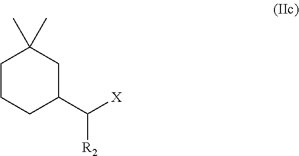
in the form of any of its stereoisomers or a mixture thereof,
wherein
X is a thiol group, alcohol group or primary amine group, and
R² a hydrogen atom or a C₁₋₄ alkyl group;
and
the compound of formula (I) is of formula (Ic) 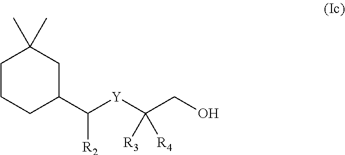
in the form of any of its stereoisomers or a mixture thereof,
wherein
Y is a sulfur atom, an oxygen atom or a NH group, and
each R², R³, R⁴ represent, when taken separately, independently from each other, a hydrogen atom or a C₁₋₄ alkyl group.

4. The process according to claim 1, wherein X is an alcohol group and Y is an oxygen atom.

5. The process according to claim 1, wherein R² represents a methyl group.

6. The process according to claim 1, wherein each R³ and R⁴ represent a methyl group.

7. The process according to claim 1, wherein the heterogeneous acidic catalyst is an aluminosilicate catalyst.

8. The process according to am claim 1, wherein the heterogeneous acidic catalyst is a zeolite or a clay.

9. The process according to claim 8, wherein the zeolite is a large pore zeolite.

10. The process according to a claim 1, wherein the heterogeneous acidic catalyst is a hydrophobic zeolite.

11. The process according to claim 1, wherein the heterogeneous acidic catalyst is a zeolite having a FAU topology.

12. The process according to a claim 1, wherein the heterogeneous acidic catalyst is a dealuminated ultrastable Y-type (USY) zeolite.

13. The process according to claim 1, wherein the silicon:aluminum ratio is in the range between 5:1 and 350:1.

14. The process according to claim 1 used for the preparation of a compound of formula (IV) 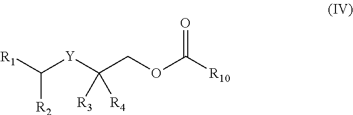
in the form of any of its stereoisomers or a mixture thereof, wherein
Y is a sulfur atom, an oxygen atom or a NH group, and
R₁ represents a C₁₋₁₂ alkyl, C₂₋₁₂ alkenyl, a C₅₋₁₂ cycloalkyl or C₅₋₁₂ cycloalkenyl group, each optionally substituted by 1 to 3 C₁₋₄ alkyl groups; and
each R₂, R₃, R₄ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; a C₁₋₄ alkyl group, a C₂₋₄ alkenyl group, a C₃₋₆ cycloalkyl group; a C₁₋₃ alkyl group, a C₂₋₃ alkenyl group, a C₃₋₅ cycloalkyl group; an ethyl group, a vinyl group, a cyclopropyl or a cyclopentyl group; and
the compound of formula (IV) is formed by reacting the compound of formula (I) with a compound of formula Z—C(O)—R₁₀, wherein Z is a R₁₀—C(O)—O group, thiol group, chlorine atom, alcohol group or amine group.

15. The process according to claim 14, wherein the compound of formula (IV) is of formula (IVa) 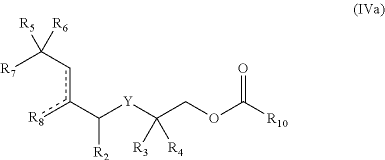
in the form of any of its stereoisomers or a mixture thereof, wherein
one dotted line is a carbon-carbon single or double bond and the other is a carbon-carbon single bond; and
Y is a sulfur atom, an oxygen atom or a NH group, and
each R₂, R₃, R₄, R₅, R₆ and R₇ represent, when taken separately, independently from each other, a hydrogen atom or a C₁₋₄ alkyl group; and
R₈ represents a C₁₋₄ alkyl group; or
R₇ and R₈ are linked to each other and form a C₅₋₇ cycloalkyl or cycloalkenyl group; and
R₁₀ represents a C₁₋₆ alkyl group, a C₂₋₆ alkenyl group, a C₃₋₆ cycloalkyl group; a C₁₋₄ alkyl group, a C₂₋₄ alkenyl group, a C₃₋₆ cycloalkyl group; a C₁₋₃ alkyl group, a C₂₋₃ alkenyl group, a C₃₋₅ cycloalkyl group; an ethyl group, a vinyl group, a cyclopropyl or a cyclopentyl group; and
the compound of formula (IVa) is formed by reacting the compound of formula (Ia) with a compound of formula Z—C(O)—R₁₀, wherein Z is a R₁₀—C(O)—O group, thiol group, chlorine atom, alcohol group or amine group.

16. The process according to claim 14, wherein the compound of formula (IV) is of formula (IVc) 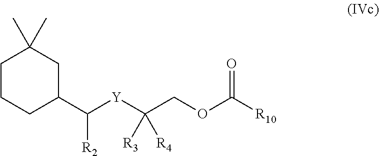
in the form of any of its stereoisomers or a mixture thereof, wherein
Y is a sulfur atom, an oxygen atom or a NH group, and
each R₂, R₃ and R₄ represent, when taken separately, independently from each other, a hydrogen atom or a C₁₋₄ alkyl group; and
R₁₀ represents a C₁₋₆ alkyl group, a C₂₋₆ alkenyl group, a C₃₋₆ cycloalkyl group; a C₁₋₄ alkyl group, a C₂₋₄ alkenyl group, a C₃₋₆ cycloalkyl group; a C₁₋₃ alkyl group, a C₂₋₃ alkenyl group, a C₃₋₅ cycloalkyl group; an ethyl group, a vinyl group, a cyclopropyl or a cyclopentyl group; and
the compound of formula (IVc) is formed by reacting the compound of formula (Ic) with a compound of formula Z—C(O)—R₁₀, wherein Z is a R₁₀—C(O)—O group, thiol group, chlorine atom, alcohol group or amine group.

17. The process according to claim 1, wherein the silicon:aluminum ratio is in the range between 15:1 and 300:1.

18. The process according to claim 14, wherein R₁₀ represents an ethyl group.

19. The process according to claim 15, wherein R₁₀ represents an ethyl group.

20. The process according to claim 16, wherein R₁₀ represents an ethyl group.
